(19) United States
(12) Patent Application Publication
(10) Pub. No.: US 2005/0256464 A1
(43) Pub. Date: Nov. 17, 2005

(54) Apparatus for washing the colon

(76) Inventor: Gilbert Pallas, Lavardac (FR)

Correspondence Address: YOUNG & THOMPSON, 745 SOUTH 23RD STREET, 2ND FLOOR, ARLINGTON, VA, 22202, US

(21) Appl. No.: 10/510,687

(22) PCT Filed: Apr. 11, 2003
(86) PCT No.: PCT/FR03/01171

§ 371 Date: May 19, 2005

(30) Foreign Application Priority Data
Apr. 11, 2002 (FR) 02/04497

(51) Int. Cl. A61M 1/00

(52) U.S. Cl. 604/319

(57) ABSTRACT

An apparatus for peroperative cleansing of the colon, includes a cylindrical body for irrigation with a cleansing liquid, the distal end of the body being provided with a strainer, elements for loosening solid fecal matter, and suction elements for sucking up the cleansing liquid and the loose fecal matter. The cylindrical irrigation body includes: two flexible coaxial tubes 2, 3 having the length of the colon and separated by a gap 4, the outer tube 2 being adaptable to the shape of the colon, the inner tube 3 serving for draining the cleansing liquid and the fecal matter, and the gap 4 between the two tubes providing a passage for the cleansing liquid; and a rigid toroidal part, which part forms an introducer element 1 having sealing membranes and attached to the colon, and in which the two coaxial tubes 2, 3 slide.

The invention relates to the field of colon surgery.

More particularly, the invention consists of a colon cleansing apparatus making it possible to cleanse the colon during an operation carried out on a closed colon via an open stomach.

Ideally, in colon surgery, the operation is carried out on a so-called “prepared” or clean colon, owing to the extremely septic nature of the content of the colon. Fecal matter is the seat of great proliferation of microbes, which are a potential source of infection for the tissue being operated on. Infection can spread throughout the body and lead to post-operative complications.

In order to avoid or reduce additional morbidity, it is customary to “prepare” the colon. Preparation consists in prescribing, prior to the operation, a substance which the patient must take a few days before the operation in conjunction with a diet that is free of residues.

In unfavorable cases, where it has not been possible to prepare the patient, due either to a deterioration in the patient's state of health, or to an emergency, the technique used at present consists in sectioning the appendix or the caecum and introducing a catheter to carry out anterograde drainage of the colon by instilling a physiological liquid which passes through the colon and is collected at the anus or at the section in the colon. That technique requires the colon to be massaged in order to move the fecal matter by making it slide, which often means that the surgeon has to dissect the left hand bend in the colon because, anatomically, it is high up and difficult to reach. Such actions add risk to the operation.

U.S. Pat. No. 5,443,445 describes a peroperative colon cleansing device, constituted by a drain tube terminating at its distal end by a screen or strainer with holes in it making it possible to prevent the invagination of the bowel, said tube receiving through a slot a lateral tube irrigated by a washing solution and receiving through a second slot upstream of the washing tube, an evacuation tube which works by suction to evacuate the washing solution and the fecal matter loosened by means of an ultrasound device located at the proximal end of the tube. The drain tube is secured by clips to the part of the colon that is to be cleansed. Nevertheless, with that device it is possible to wash only that part of the colon which is near to the strainer, and under no circumstances can it wash the entire colon, given that the strainer is not movable. Moreover, there is no mechanical sweeping to move the fecal matter adhering to the surface of the mucous membrane in the colon.

The object of the invention is to supply a drain tube which can be inserted into the colon and can enable its various parts to be rigorously cleansed by progressing segment by segment.

Given the length of the colon, which is about 1.50 meters (m) long, and therefore the length of the tube, the irrigation and evacuation circuits need to be independent of each other up to the strainer, in order to prevent the irrigation and evacuation liquids from mixing. Any such mixing would lead to reduced effectiveness and would waste time, on account of the dead volume due to the great length of the tube.

A second object of the invention is to provide equipment enabling cleansing to be effective and fast.

The problem with cleansing tubes is the suction at the distal end which is due to the shape of the strainer and to the residual pressure, even when the suction has been turned off. This phenomenon leads to permanent blocking of the tube and therefore to ineffective functioning of said tube.

The existing means consists in irrigating again, which increases the duration of the operation.

The apparatus of the invention remedies the above-mentioned drawbacks.

A further object of the invention is to provide an apparatus that is perfectly leaktight in order to avoid the peritoneum from being seeded with microbic germs coming from the fecal matter.

In order to achieve this, the invention consists of an apparatus for peroperative cleansing of the colon, the apparatus comprising a cylindrical body for irrigation with a cleansing liquid, its distal end having a strainer, means for loosening solid fecal matter, and means for sucking up the cleansing liquid and the loose fecal matter, the apparatus being characterized in that the cylindrical irrigation body comprises:
two flexible coaxial tubes having the length of the colon and separated by a gap, the outer tube being adaptable to the shape of the colon, the inner tube serving for draining the cleansing liquid and the fecal matter, and the gap between the two tubes providing a passage for the cleansing liquid; and
a rigid toroidal part forming an introducer element having sealing membranes and attached to the colon, and in which the two coaxial tubes slide.

At its distal end, the inner tube of the cylindrical irrigation body carries a strainer having a mechanical sweeping device.

The inner tube also includes a rotary device for grinding fecal matter.

In one embodiment, the grinding device is a rotary knife having a central drive shaft and pressed against a grid.

In a preferred embodiment, the strainer is a flexible cylindrical body terminated by a spherical cap, having a total length lying in the range 6 centimeters (cm) to 10 cm and with numerous circular or oval holes, the first hole being located at a distance lying in the range 2 cm to 3 cm from the knife, the diameter of the holes increasing in size from 2 millimeters (mm) to 15 mm on going from the proximal portion to the end of the strainer.

Preferably, the outer tube has an outside diameter lying in the range 18 mm to 24 mm, the inner tube has an inside diameter lying in the range 12 mm to 18 mm and the strainer has an outside diameter lying in the range 18 mm to 24 mm.

The cleansing liquid, which may be sterile physiological serum, for example, is supplied at a high rate lying in the range 0.5 litres/minute (L/m) to 1 L/m.

In order to satisfy the looked-for characteristics, the inner tube and the strainer are made of flexible plastics material, and the outer tube is made of a malleable material.

In a variant of the embodiment, the mechanical sweeping device is constituted by one or more rows of annular brushes.

Preferably, the suction means comprise a tube connected to a wall vacuum source and opening out laterally into the cylindrical irrigation body at a control handle.

In a preferred embodiment, the irrigation, loosening and suction means are operated at will via mutually-independent voluntary controls.

The irrigation means includes a foot-operated control pedal, the loosening means include an operating handle with switch, for keeping switched on, and the suction means include an operating handle with vent closure.

Preferably, the apparatus is provided with a pressurized safety appliance in the event forced irrigation is required, since said safety appliance ensures that both coaxial tubes are put to atmospheric pressure.

The apparatus has the advantage of enabling the whole colon to be cleansed because the long outer tube can progress along the colon, adapting itself to the shape of the colon. Two coaxial tubes are used so that the irrigation fluid and suction fluid are not mixed up together.

Another advantage of the invention is that it avoids the suction phenomenon, due to the fitted shape of the strainer and by eliminating the residual pressure remaining in the strainer when suction at the strainer has been stopped.

The inside of the colon is lined with a mucous membrane comprising villi. This layer, or tunic, is extremely flexible, fragile, and stretchable, and it has the capacity to slide relative to the other tunics in the colon (the muscular and the serosa). Occasionally, the colon wall itself is very thin and flexible and can itself become invaginated like the mucous membrane on a suction hole, thereby leading to a suction phenomenon.

The shape of the strainer is fundamental for preventing any contact between the mucous membrane and the knife. This requires a minimum safe distance of 2 cm between the first holes in the strainer and the knife. The diameter of the holes should increase on going from the proximal portion of the knife to the distal portion, so that the smallest holes make it possible to hold the mucous membrane of the colon stationary and to limit invagination into the largest holes.

Moreover, when suction is stopped, residual pressure remaining in the strainer is eliminated by putting an additional tube into place, which tube opens out into a collection vessel. It is thus possible to create an air inlet so that the collection vessel can be put to atmospheric pressure.

Eliminating invagination both saves time in comparison with the traditional technique and reduces cleansing time to less than 30 minutes.

The apparatus of the invention allows for effective cleansing by using a mechanical sweeping device having one or more annular brushes which are vital for moving the fecal matter adhering to the surface of the mucous membrane in the colon by means of mechanical back-and-forth movements.

Lastly, the apparatus ensures total leaktightness, which prevents the peritoneum from being seeded by microbic germs from the fecal matter, due to an introducer attached to the opening in the colon and provided with a cover connecting it in leaktight manner to the control handle. The cleansing device is movable relative to the introducer secured to the colon.

Other characteristics and advantages of the invention appear in the detailed, but non-limiting, description below.

The following description refers to the accompanying drawings which show, in a non-limiting manner, an embodiment of an apparatus intended for the preoperative cleansing of the colon.

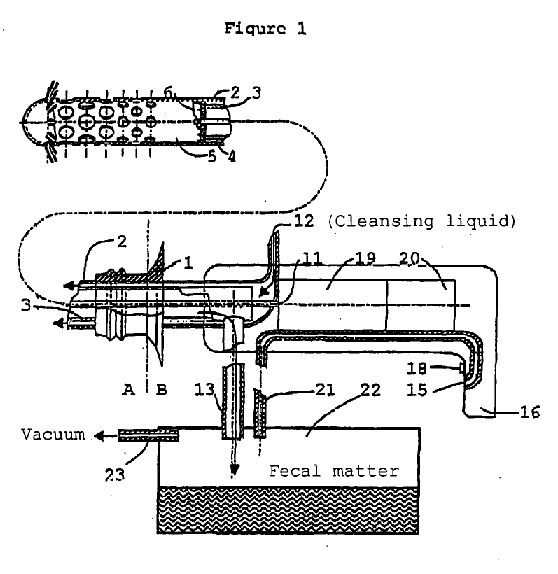

FIG. 1 is a diagram of a device of the invention for cleansing the colon.

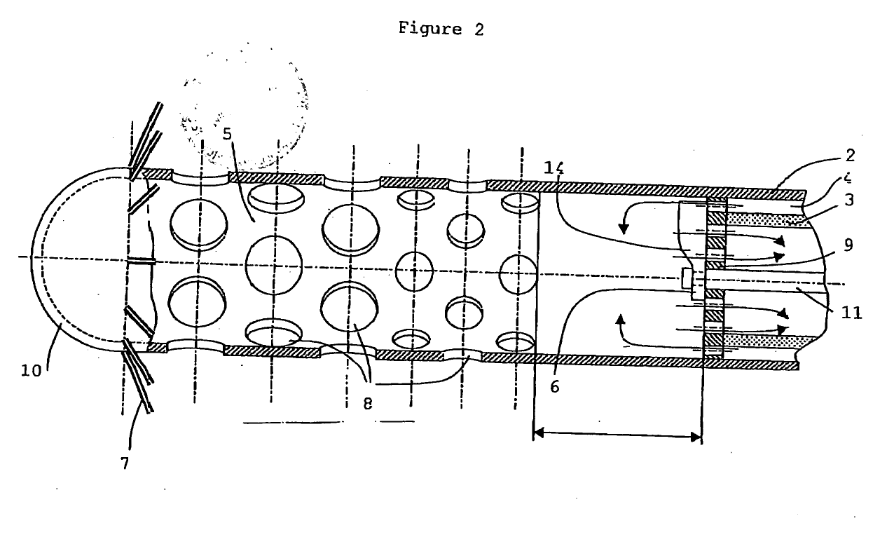

FIG. 2 is a detailed view of the distal end of FIG. 1.

The apparatus shown in FIG. 1 consists of a part A for insertion into the patient's colon and a part B which remains on the outside the patient.

Part A comprises an introducer constituted by a rigid toroidal part. The introducer is provided with sealing membranes. It is attached to the place where the incision is made in the colon. It enables the cleansing device to be inserted, which device comprises two coaxial tubes 2 and 3 that are at least 80 cm long and whose distal end is fitted with a strainer 5 having a ring-shaped brush 7. A cover (not shown) links the introducer to the handle for manually displacing the two coaxial tubes in translation inside the colon. The cover is for isolating the outer tube from the operator's hand.

The outer tube 2, having an outside diameter lying in the range 18 mm to 24 mm, is made in a malleable plastics material so that it can be adapted to fit the shape of the colon.

The inner tube 3, having an inside diameter lying in the range 12 mm to 18 mm, is flexible, limp, and shapeless and with little elastic memory, due to its ringed spiral design.

The gap 4 between the two tubes is for circulating the cleansing liquid constituted by physiological serum at body temperature.

As is shown more clearly in FIG. 2, the distal end of the two tubes is fitted with a strainer 5 with circular or oval holes 8 having diameters lying in the range 2 mm to 15 mm, which diameters increase in size on going from the proximal portion to the end of the strainer. The strainer is in the form of a cylindrical body, having the same outside diameter as the outer tube, terminated in its distal portion by a spherical cap 10 having a total length lying in the range 6 cm to 10 cm.

Inside the slot in the inner tube, a rotary knife 6 having a flexible central drive shaft 11 and pressed against a grid 9 makes it possible to move the fecal matter which becomes stuck to the proximal orifices in the strainer and to cut it up into pieces against the grid which it touches.

The knife 6 is located at a safe distance lying in the range 2 cm and 3 cm from the proximal orifices in the strainer in order to prevent any contact between the mucous membrane of the colon and the knife.

The knife 6 has a blade 14 with a large pitch angle close to 45°. The knife converts dehydrated, compact, soft, or even liquid fecal matter into a homogenous, liquidized medium which can be more easily evacuated.

For reasons relating to sterility, the entire part A is, preferably, for single use only.

Part B outside the patient includes a handle 16 having a switch 18 and containing or supporting a motor 19 powered from a storage battery 20 and driving the shaft 11. The switch 18 needs to be held on, in order to actuate the rotary knife 6.

The handle 16 has a vent 15, which can be closed fully or in part, enabling the operator to modulate the intensity of the vacuum inside the strainer 5. An air inlet tube 21 connected to a collection vessel 22 opens out from the handle through the vent 15.

The cleansing liquid 12 is fed at a high rate lying in the range 0.5 L/m to 1 L/m from a receptacle, such as a bag or a bottle. This is carried out by means of a foot-operated pedal (not shown).

The evacuation tube 13 opens into the handle. Suction is obtained by closing the vent 15 manually. The collection vessel is connected by a tube 23 to a wall-mounted vacuum outlet.

The colon cleansing apparatus of the invention works as follows:

After proceeding with the exeresis of the occlusive tumour, the operator inserts the proximal end of the introducer 1 and attaches it to the colon by a stitch. The operator then slides the two coaxial tubes 2 and 3 into the introducer 1 and extends the protective cover as far as the control handle 16.

The first step consists in suction, which is obtained by closing the vent 15.

The second step consists of irrigation, which is activated by the foot-operated control. When the colon is full, the operator proceeds to suction once again.

The maneuver consisting in irrigation followed by suction needs to be repeated until the liquid being collected is as transparent as water, without any visible residues.

If there is a blockage, i.e. if suction does not work when the colon is full, the operator turns on the switch 18 to actuate the rotary knife 6.

When the fecal matter adheres or is viscous, a back-and-forth movement in translation can be carried out by part A in order to complete cleansing of the surface of the mucous membrane of the colon.

As soon as one segment of colon is clean, the operator uses the apparatus on a new, uncleansed segment, and so on until the whole colon is clean.

1. An apparatus for peroperative cleansing of the colon, the apparatus comprising a cylindrical body for irrigation with a cleansing liquid its distal end having a strainer, means for loosening solid fecal matter, and means for sucking up the cleansing liquid and the loose fecal matter, the apparatus being characterized in that the cylindrical irrigation body comprises:
two flexible coaxial tubes (2, 3) having the length of the colon and separated by a gap (4), the outer tube (2) being adaptable to the shape of the colon, the inner tube (3) serving for draining the cleansing liquid and the fecal matter, and the gap (4) between the two tubes providing a passage for the cleansing liquid; and
a rigid toroidal part forming an introducer element (1) having sealing membranes and attached to the colon, and in which the two coaxial tubes (2, 3) slide.

2. An apparatus according to claim 1, characterized in that the inner tube of the cylindrical irrigation body carries at its distal end a strainer (5) having a mechanical sweeping device.

3. An apparatus according to claim 2, characterized in that the inner tube (3) includes a rotating device for grinding fecal matter.

4. An apparatus according to claim 3, characterized in that the grinding appliance is a rotary knife (6) having a central drive shaft (11) and pressed against a grid (9).

5. An apparatus according to claim 2, characterized in that the strainer (5) is a flexible cylindrical body terminated by a spherical cap, having a total length lying in the range 6 cm to 10 cm and with numerous circular or oval holes (8), the first hole being located at a distance lying in the range 2 cm to 3 cm from the knife, the diameter of the holes increasing in size from 2 mm to 15 mm on going from the proximal portion to the end of the strainer.

6. An apparatus according to claim 2, characterized in that the outer tube (2) has an outside diameter lying in the range 18 mm to 24 mm, the inner tube (3) has an inside diameter lying in the range 12 mm to 18 mm and the strainer (5) has an outside diameter lying in the range 18 mm to 24 mm.

7. An apparatus according to claim 1, characterized in that the supply (12) of cleansing liquid is carried out at a high rate lying in the range 0.5 L/m and 1 L/m.

8. An apparatus according to claim 2, characterized in that the inner tube (3) and the strainer (5) are embodied in a flexible plastics material, the outer tube in malleable material.

9. An apparatus according to claim 2, characterized in that mechanical sweeping device is constituted by one or more rows of annular brushes (7).

10. An apparatus according to claim 1, characterized in that the suction means comprise a tube connected to a wall vacuum source and opening out laterally into the cylindrical irrigation body at a control handle.

11. An apparatus according to claim 11, characterized in that the irrigation, loosening and suction means are operated at will via mutually-independent voluntary controls.

12. An apparatus according to claim 11, characterized in that the irrigation means comprise a foot-operated control pedal, the loosening means comprise an operating handle with switch (18) to be maintained in a blocked position and the suction means comprise an operating handle with vent closure (15).

13. An apparatus according to claim 12, characterized in that it is provided with a pressurized safety appliance (21) in the event forced irrigation is required, thereby ensuring that both coaxial tubes are put to atmospheric pressure.
